(19) United States
(12) Patent Application Publication
(10) Pub. No.: US 2025/0153162 A1
(43) Pub. Date: May 15, 2025

(54) DUAL-COMPONENT SYSTEM AND DEVICE FOR PROCESSING AND ANALYZING POLYMERASE CHAIN REACTIONS WITHIN A CARTRIDGE AND CORRESPONDING METHODS OF USE

(71) Applicant: InBios International, Inc., Seattle, WA (US)

(72) Inventors: James William Needham, Seattle, WA (US); Suvro Datta, Seattle, WA (US); Hillary Cartwright, Seattle, WA (US); Sharon Choi, Seattle, WA (US)

(21) Appl. No.: 18/942,193

(22) Filed: Nov. 8, 2024

Related U.S. Application Data
(60) Provisional application No. 63/597,611, filed on Nov. 9, 2023.

(51) Int. Cl. B01L 3/00; B01L 7/00; C12Q 1/686

(52) U.S. Cl. CPC B01L 3/502; B01L 7/52; C12Q 1/686; B01L 2200/026; B01L 2200/16; B01L 2300/048; B01L 2300/0883

(57) ABSTRACT

A two-component system and device for efficient analysis of Nucleic Acid Amplification Tests (NAAT) on a cartridge that is portable, low cost, delivers rapid results with direct sample application, minimal sample volume, and has a user-friendly interface. The system and device can be used to perform NAAT reactions, either thermal or isothermal and either in single-plex or multi-plex, on a sample within a single-use test cartridge that is integrated for use with a reusable separately housed amplification module and analysis module to generate test results.

### FIELD OF THE INVENTION

Described herein is an integrated two-component system and device for efficient analysis of Nucleic Acid Amplification Tests (NAAT) on a cartridge that is portable, low cost, delivers rapid results with direct sample application, minimal sample volume, and has a user-friendly interface. The system and device can be used to perform NAAT reactions, either thermal or isothermal and either in single-plex or multi-plex, on a sample within a single-use test cartridge that is integrated for use with a reusable separately housed amplification module and analysis module to generate test results. The system and device may be used at point-of-care or over-the-counter settings to permit the streamlined testing and interpretation of test results. Integrated software may provide the user with simple feedback and test interpretation results. The system and device have minimized the device footprint, enhanced thermal cycling times, and permits the direct insertion of a first component test cartridge into the second component which provides for nucleic acid amplification and analysis. The NAAT may comprise, but is not limited to, polymerase chain reaction (PCR), reverse transcription PCR (RT-PCR), and real-time RT-PCR (rRT-PCR) tests.

### BACKGROUND

There are over one billion infections in the U.S. each year, many of which are treated incorrectly due to inaccurate or delayed diagnostic results. Many known point of care (POC) tests have poor sensitivity (30-70%), while the more highly sensitive tests, such as those involving the specific detection of nucleic acids or molecular testing associated with a pathogenic target, are only available in laboratories. Thus, almost all of the current molecular diagnostics testing is practiced in centralized laboratories. Known devices and methods for conducting laboratory-based molecular diagnostics testing, however, conventionally requires trained personnel, regulated infrastructure, and expensive, high throughput instrumentation. Known laboratory instrumentation is often purchased as a capital investment along with a regular supply of consumable tests or cartridges. Known high throughput laboratory equipment generally processes many (96 to 384 and more) samples at a time, therefore central lab testing is done in batches. Known methods for processing typically include processing all samples collected during a time period (e.g., a day) in one large run, with a turn-around time of hours to days after the sample is collected. Moreover, such known instrumentation and methods are designed to perform certain operations under the guidance of a skilled technician who adds reagents, oversees processing, and moves sample from step to step. Thus, although known laboratory tests and methods are very accurate, they often take considerable time, and are very expensive.

There are limited testing options available for testing done at the point of care (“POC”), or in other locations outside of a laboratory. Known POC testing options tend to be single analyte tests with low analytical quality. These tests are used alongside clinical algorithms to assist in diagnosis, but are frequently verified by higher quality, laboratory tests for the definitive diagnosis. Thus, neither consumers nor physicians are enabled to achieve a rapid, accurate test result in the time frame required to “test and treat” in one visit. As a result doctors and patients often determine a course of treatment before they know the diagnosis. This has important consequences: antibiotics are either not prescribed when needed, leading to infections; or antibiotics are prescribed when not needed, leading to new antibiotic-resistant strains in the community. Moreover, known systems and methods often result in diagnosis of severe viral infections, such as H1N1 swine flu, too late, limiting containment efforts. In addition, patients lose much time in unnecessary, repeated doctor visits.

There is an unmet need for improved devices, systems, and methods for molecular diagnostic testing. In particular, a need exists for an affordable, easy-to-use test that will allow healthcare providers and patients at home to diagnose infections accurately and quickly so they can make better healthcare decisions.

### SUMMARY OF THE INVENTION

Described herein is an integrated two-component system and device for efficient analysis of Nucleic Acid Amplification Tests (NAAT) on a cartridge that is portable, low cost, delivers rapid results with direct sample application, minimal sample volume, and has a user-friendly interface. The system and device can be used to perform NAAT reactions, either thermal or isothermal and either in single-plex or multi-plex, on a sample within a single-use test cartridge that is integrated for use with a reusable separately housed amplification module and analysis module to generate test results. The system and device may be used at point-of-care or over-the-counter settings to permit the streamlined testing and interpretation of test results. Integrated software may provide the user with simple feedback and test interpretation results. The system and device have minimized the device footprint, enhanced thermal cycling times, and permits the direct insertion of a first component test cartridge into the second component which provides for nucleic acid amplification and analysis.

The system and device uniquely provide, among other features, a single-use first testing cartridge first component comprising a first part ‘A’ and a second part ‘B’ that together form a communication network of interior chambers and pathways for complete processing of a sample. The complete processing of the test sample takes place within the test cartridge, including lysis of the sample material, introduction of NAAT reagents, amplification (denaturation, annealing, and extension, e.g., by thermal cycling) within the cartridge reaction well, and test sample analysis. Notably, the processing of the test sample within the cartridge first component is aided by the separable second component which comprises an amplification module and an analysis module within a housing.

The two components of the system and device as described herein are each uniquely adapted to the other to facilitate efficient and accurate processing of test samples using minimal volumes of sample, buffer, and reagent materials. The first component test cartridge is specially designed to maximize the utility of the processing spaces provided therein and, particularly, to provide for processing of test samples with the aid of the second component amplification and analysis modules. The first component test cartridge may be specially designed to be wholly or partly transparent and permit passage of excitation energy or light energy through the test cartridge. The first component test cartridge may be specially designed to allow introduction of excitation energy or light energy along a lateral side or bottom edge of the test cartridge. The first component test cartridge may be specially designed to allow analysis of the sample reaction volume in the cartridge reaction well along a lateral side or bottom edge of the test cartridge. The design of each component is especially adapted for integration with the other. Integrated design features of the first component and the second component permit insertion and withdrawal of the test cartridge and locate the cartridge reaction well directly facing, opposite or between, one or more thermal cycling amplification components housed within the second component. Additional integrated design features of the first component and the second component permit introduction of excitation energy or light energy along a lateral side or bottom edge of the test cartridge into the sample reaction volume in the cartridge reaction well, for example, using an LED holder along with an excitation filter holder and concentrator lens, and analysis of the sample reaction volume in the cartridge reaction well along a lateral side or bottom edge of the test cartridge, for example, using a photodiode holder along with an emission filter holder and focusing lens.

In one preferred embodiment, the interior chambers and pathways of the test cartridge comprise, a buffer control area in contact with a buffer region, a buffer region in communication with a sample receiving port, a sample receiving port and buffer mixing zone in communication with a siphon U-turn, a siphon U-turn in communication with a drawing tunnel, and a drawing tunnel in communication with a reaction well, and a reaction well that is also in communication with a vent. Also housed within the reaction well of the cartridge may be reagent materials that are pre-loaded. The reaction well of the cartridge can be broad and shallow; thus providing for a maximally spread thin layer of sample reaction volume. A broad and shallow reaction well provides a maximum surface area exposure to one or more exterior heating and cooling elements involved in thermal cycling (located in the second component amplification module) to facilitate rapid heating and cooling of the sample reaction volume, thus expediting thermal PCR reactions within the minimal reaction volume necessary. In one embodiment, the reaction well of the cartridge can comprise a narrow zigzag, or tortuous, course or pathway structure of the reaction well to further facilitate processing of minimal reaction volumes.

The device also uniquely provides for a generally flattened testing cartridge configuration that encases the reaction well and provides for broad flattened exterior-facing cartridge surfaces for thermal contact with one or more exterior heating and cooling elements involved in thermal cycling (located in the second component amplification module). The system also uniquely provides for the optional sandwiching of the flattened test cartridge between multiple exterior thermal cycling elements to maximize thermal transfer between the minimal reaction volume within the test cartridge and the exterior thermal cycling elements.

In one preferred embodiment, the system also provides for a generally flattened test cartridge configuration that permits transverse, lateral, or sideways excitation of the minimal reaction volume in the reaction well, and transverse, lateral, or sideways detection of signals generated in the reaction volume in the reaction well by one or more exterior excitation and detection elements (located in the second component analysis module).

The test cartridge can be comprised of materials such as, polycarbonate, etc. The test cartridge may be wholly or partly transparent in order to permit passage of excitation energy or light energy as well as detection of signals generated in the reaction volume in the reaction well.

### BRIEF DESCRIPTION OF THE DRAWINGS

FIG. 1A depicts an embodiment of sample specimen collection. FIG. 1A depicts a flocked swab that is to be rotated within the nasal cavity to collect a mucus specimen sample. FIG. 1B depicts a first component test cartridge embodiment with a sample receiving port, a bar code, and a reaction well. FIG. 1C depicts an embodiment of the final form factor of a device disclosed herein wherein the first component test cartridge of FIG. 1B is inserted into the second component comprising an amplification module and an analysis module within a housing. FIG. 1C further depicts an embodiment of the second component wherein the housing includes an external surface display screen.

FIG. 2 shows an incomplete embodiment of components of the device described herein. In this incomplete embodiment two Peltier heating and cooling blocks on either side of a first component test cartridge. In this configuration, the test cartridge, including the test cartridge reaction well, is sandwiched between the two Peltier heating and cooling blocks.

FIGS. 3A and 3B show the temperature (Celsius) and voltage (V) over time (seconds) obtained using an embodiment similar to that shown in FIG. 2. FIG. 3A shows multiple cycles of heating and cooling and includes oval marking indicating an area of interest that is enlarged to provide greater detail in FIG. 3B. FIG. 3B shows that the temperature control obtained using two Peltier heating and cooling blocks on either side of a first component test cartridge provided control over about 20 seconds with a variation well-within ±° 1C.

FIG. 4 is an open view of an incomplete embodiment of a device 300 as described herein. As can be seen a partial first component test cartridge 100 that is located in a second component 200 that includes two Peltier heating and cooling blocks 202 on either side of the test cartridge 100. Each Peltier heating and cooling block 202 comprises a Peltier heat and cool surface 204 and a fan 206 and a heat sink 208. Wires can also be seen that provide power to the device 300 and that permit communication of information, including data, with the device 300 from at least one other source (not shown).

FIGS. 5A and 5B show the temperature (Celsius) over time (seconds) obtained using the embodiment shown in FIG. 4. FIG. 5A shows multiple cycles of heating and cooling and includes oval marking indicating an area of interest that is enlarged to provide greater detail in FIG. 5B. FIG. 5B shows that the temperature control obtained using two Peltier heating and cooling blocks on either side of a first component test cartridge provided control well within +0.5° C. of the 59° C. set point temperature over a 20 second time period and this is reproducibly generated over a standard 40-cycle PCR run.

FIG. 6 is an example of a first component test cartridge microfluidic chip embodiment which provides proof of concept that PCR reactions in a microfluidic chip can successfully produce target amplicon and that fluorescence is possible in the microfluidic chip using methods described herein. The first component test cartridge microfluidic chip shown here utilizes a microscope and coverslip design which is easy to manufacture and inexpensive.

FIG. 7 provides an example interior view of a part ‘A’ of a first component test cartridge 100 that provides controlled fluidic flow in the test cartridge 100. The test cartridge 100 comprises a part ‘A’ and a part ‘B’ which is added, or attached (as shown in FIG. 8), to cover and seal the broad interior cavity surface face 118 of part ‘A’. As shown, part ‘A’ of the first component test cartridge 100 includes a buffer control area 102, a buffer region 104, a sample receiving port 106, a buffer mixing zone 108, a siphon U-turn 110, a drawing tunnel 112, a reaction well 114, and a vent 116. Also, included is the interior facing surface 118 which, together with part ‘B’ of a first component test cartridge, forms cartridge 100.

FIG. 8 shows part ‘A’ of a first component test cartridge 100A of FIG. 7 with a part ‘B’ of a first component test cartridge, which is shown here as a cover 100B comprising a thin sheet of material which is attached to the surface of the broad interior cavity surface face 112 of part ‘A’ 100A. The cover material may comprise polycarbonate and may be clear.

FIG. 9. The primers and probes selected for HIV-1 and HIV-2 are indicated in the table of FIG. 9. These designed primers sets detect conserved regions of HIV-1's polymerase gene (pol) and HIV-2's long terminal repeat (LTR). The HIV-1 genome organization is shown with protein coding regions shown as gray boxes; polyprotein domain junctions depicted as solid vertical lines; and gene start and end sites numbered according to NL4-3is. It is noted that this HIV-1 genome organization image is copied from FIG. 1a of Watts J M, et al. Architecture and Secondary Structure of an Entire HIV-1 RNA Genome. Nature. 460, 711-716 (2009).

FIG. 10. The primers and probes selected for HBV target the S-gene and are indicated in the table of FIG. 10. The image depicting the linear arrangement of overlapping ORFs and viral transcripts is adapted from FIG. 1B of Lamontagne J. Hepatitis B virus and microRNAs: Complex interactions affecting hepatitis B virus replication and hepatitis B virus-associated diseases. World Journal of Gastroenterology. 21, 7375-7399 (2015). As depicted, the transcripts and ORF are aligned with each other according to genomic location and are to scale, and colored boxes on transcripts indicate proposed miRNA target sites.

FIG. 11. The primers and probes selected for HCV are indicated in the table of FIG. 11. The primers and probes selected for HCV target the conserved 5′ UTR region of this RNA virus. The image depicting stem-loop structures is copied from FIG. 1 of Fricke M. Conserved RNA secondary structures and long-range interactions in hepatitis C viruses. RNA Journal. 21, 1219-1232 (2015). This image provides an overview of previously known (black) and novel (gray) RNA stem-loop structures of the HCV RNA genome. (A) 5′ UTR and core region SLV and SLVI of the viral plus-strand. (B) CRE region and 3′ UTR of the viral plus-strand. (C) 3′ End of the viral minus-strand. (D) 5′ End of the viral minus-strand.

FIG. 12 is an image of PCR product generated using a microfluidic cartridge of the device disclosed herein and run on a gel to establish proof-of-principle. FIG. 12 shows that a sample influenza B matrix target was successfully amplified in both the Bio-Rad CFX96 and a device as disclosed herein. A simple PCR assay was created to target the influenza B (flu B) matrix gene. The flu B amplicon PCR proof-of-principle testing detects an amplicon of length 139 bp and is amplified using two forward primers, one reverse primer and a FAM labelled probe. Labels are provided on the gel image to identify the 100 bp and 10 kb lengths. The abbreviations used in FIG. 12 include: no template control (NTC), the commercially available Bio-Rad CFX96 machine (CFX), and the presently disclosed device, referred to here as Gene Detect (GD).

FIG. 13 shows fluorescence detection on a microfluidic chip using the microfluidic chip of FIG. 6 in 0 nM (water), ˜70 nM and 500 nM FAM.

FIG. 14 provides a gel image that compares the presently disclosed device performance with the commercially available Bio-Rad CFX device. The influenza B PCR assay was performed with varying cycle counts, ranging from 5, 10, 15, 20, 25, 30, 35 and 40 cycles in each device. The PCR products that were generated using the CFX and a device as described herein, referred to here as Gene Detect or (GD), were analyzed side-by-side via a 3% agarose gel to qualitatively compare the results as summarized in FIG. 14. No template control (NTC) was performed for 40 cycles and are shown in lanes 17 and 18.

FIG. 15 is a graph chart showing serial dilutions of FAM fluorophore using the optics suitable for use with a device as described herein. It is shown that titrations of the FAM fluorophore provide a linear relationship with the measured voltage.

FIG. 16 provides a gel image of PCR product from the HBV PCR reactions from the CFX and a device as described herein, referred to here as Gene Detect or (GD), visualized via 3% agarose gel with reactions for HBV. HBV single plex reactions are shown.

FIG. 17 provides a gel image of PCR product from the HCV and HIV-1 PCR reactions from the CFX and a device as described herein, referred to here as Gene Detect or (GD), are visualized via 3% agarose gel with reactions for HCV and HIV-1. HCV and HIV-1 single plex reactions are shown.

FIG. 18 shows the CFX amplification traces for the HBV, provided as relative fluorescence units (RFU) by cycles.

FIG. 19 shows the CFX amplification traces for the HCV and HIV-1, provided as relative fluorescence units by cycles.

FIG. 20 shows a gel image of PCR product from multiplexed testing performed on the CFX and a device as described herein, referred to here as Gene Detect or (GD). Multiplex testing for HBV, HCV and HIV was performed. The multiplex reaction worked for the detection of HBV, HCV and HIV with the CFX. However, when run on the GD, the device only showed the HBV and HCV bands corresponding to the targets when multiple target templates were added to the same reaction, indicating that additional problem-solving may be in order to improve multiplex functionality.

FIG. 21 provides an example interior view of a first component test cartridge 400A that provides controlled fluidic flow. The test cartridge 400 comprises a part ‘A’ and a part ‘B’ which is added, or attached (not shown), to cover and seal the broad interior cavity surface face 418 of part ‘A’ which may be, for example, similar to the cover 100B as shown in FIG. 8. As shown, part ‘A’ of the first component test cartridge 400 includes a buffer control area 402, a buffer region 404 which may or may comprise a buffer blister pack (not shown), a sample receiving port 406, a buffer mixing zone 408, a siphon U-turn 410, a drawing tunnel 412, a reaction well 414, and a vent 416. Also, included is the interior facing surface 418 of first component test cartridge 400A which, together with part ‘B’ of a first component test cartridge, forms cartridge 400.

FIG. 22A and FIG. 22B depict a fluorescence measurement setup 500 for exemplary components of the system and device described herein. FIG. 22A shows an incomplete embodiment of a fluorescence measurement setup for the system and device, comprising a photodiode holder 502 along with an emission filter holder 504 and focusing lens 506 located on a lateral side of the test cartridge 400A and proximal to the test cartridge reaction well 414. Also in this embodiment an LED holder 508 along with an excitation filter holder 510 and concentrator lens 512 are located on a bottom lateral side of the test cartridge 400A and proximal to the test cartridge reaction well 414. In the depicted configuration, the photodiode holder 502 along with an emission filter holder and focusing lens and the LED holder along with an excitation filter holder and concentrator lens are located on distinct separate lateral sides of the test cartridge 400A. Specifically, as depicted the photodiode holder 502 along with an emission filter holder 504 and focusing lens 506 and the LED holder 508 along with an excitation filter holder 510 and concentrator lens 512 are located on a lateral vertical side of the test cartridge 400A and on a lateral lower or bottom side of the test cartridge 400A, respectively. It is contemplated, however, that the location of the photodiode holder 502 along with an emission filter holder 504 and focusing lens 506 and the LED holder 508 along with an excitation filter holder 510 and concentrator lens 512 may be located opposite to each other on either lateral side of the test cartridge 400A or on the same lateral side of the test cartridge 400A, or in any position with respect to each other. The photodiode holder 502 along with an emission filter holder 504 and focusing lens 506 may be collectively referred to herein as an emission assembly. The LED holder 508 along with an excitation filter holder 510 and concentrator lens 512 may be collectively referred to herein as an excitation assembly. FIG. 22B provides the prototype fluorescence measurement setup 500 show in FIG. 22A.

FIG. 23 is a graph chart showing FAM concentration versus photodiode output using the fluorescence measurement setup 500 depicted in FIG. 22A and FIG. 22B for different values of LED current.

FIG. 24, like FIG. 4, is an open view of an incomplete embodiment of a system and device 300 as described herein. But here, as can be seen, a first component test cartridge 400 rather than a partial first component test cartridge 100 (as shown in FIG. 4) is located in a second component 200 that includes two Peltier heating and cooling blocks 202 on either side of the test cartridge 400. Each Peltier heating and cooling block 202 comprises a Peltier heat and cool surface 204 and a fan 206 and a heat sink 208. Wires can also be seen that provide power to the device 300 and that permit communication of information, including data, with the device 300 from at least one other source (not shown).

### DETAILED DESCRIPTION

Preferred embodiments of the two-component system and device for efficient analysis of NAAT provide an inexpensive, accessible, and patient-centered rapid thermal cycling NAAT device with direct sample application for the detection of RNA and/or DNA present in whole blood samples, or other sample types.

Design criteria ensured simpler user interaction and direct sample application. The integrated two-component system and device is also characterized by a small device footprint, enhanced thermal cycling times, and the direct insertion of a first component test cartridge into a second component to facilitate amplification and analysis of the test sample contained in the first component.

### Definitions

“Automated” is meant to indicate that a process is automatic, nonmanual, or self-operating. For example, in a preferred embodiment, the sample processing (including, for example, lysis and amplification) and analyses processes (including, for example, excitation and detection of reaction mixture products within the reaction volume) described herein are fully automated once a sample is introduced into a sample receiving port. In another embodiment, the sample processing and analyses processes described herein are fully automated once a sample is introduced into a sample receiving port and lysis buffer is added.

“Bar code” is meant to refer to either any type one-dimensional (1D) machine-readable code which is made up of a pattern of lines of varying widths which may or may not be accompanied by number, and of matrix barcode.

“Cartridge” is meant to refer to the first component of the device and system and is understood to refer to a container, cassette, or casing comprising within it a series of fluidic pathways, chambers, and/or wells and, optionally, pre-loaded components such as, for example, lysis buffer and one or more reagent materials.

“Enhanced thermal cycling times” refers to improved timing achieved in relation to thermal cycling of a particular volume of material, and the speed at which repeated variation in increasing and lowering the temperature of the particular volume of material may be achieved. “Fluidic flow” is meant to generally refer to the motion of a fluid.

“Fluorophore” refers to a molecule with fluorescence properties, i.e., which can absorb and emit photons, or particles of light, of different wavelengths.

“In communication with” refers to a direct physical connection, contact, or link between one thing and another thing.

“Insertion” or “insert” or “inserted” means, in the physical sense, to put one thing into another thing.

“Integrated” means the coordination or combination of two or more things in order to become more effective.

“Lyophilized” refers to a freeze-dried substance or a process in which water is removed from a product after is frozen and placed under a vacuum, allowing the ice to change directly from a solid to a vapor without passing through a liquid phase.

“Lysis buffer” refers to a buffer solution used for the purpose of breaking open cells for use in molecular biology.

“Maximally spread” as used herein refers to a broad, or the broadest possible, physical expansion of a reaction mixture reaction volume over a surface and/or within a well or fluid path that still permits analyses of the reaction volume.

“Microfluidic” refers to a system or pathway that manipulates a small amount of fluids using small channels with sizes ten to hundreds of micrometers, and refers to the behavior of fluids through micro-channels, and the technology of manufacturing microminiaturized devices containing chambers, wells, pathways, and tunnels through which fluids flow or are confined.

“Minimal reaction volume” refers to a small, or the smallest possible, physical amount of reaction volume (comprising sample, buffer, and reagents) needed to perform amplification and/or analyses of reaction products.

“Minimal sample volume” refers to a small, or the smallest possible, physical amount of sample volume (comprising sample and buffer) needed to conduct sample processing by the cartridge.

“Minimized device footprint” refers to a small, or the smallest possible, physical amount of physical space occupied by the second component and/or the second component and the first component of the device and system described herein.

“Pre-loaded” means to load, or put something on or in to something else, in advance and at a time removed from the time of use.

“Primer” refer to a single-stranded nucleic acid sequence that provides a starting point for DNA synthesis.

“Probe” refers to a single-stranded sequence of DNA or RNA that is used to identify specific sequences of DNA or RNA.

“Reagent” refers to a compound or mixture added to a system to start or test a chemical reaction. A reagent can be used to determine the presence or absence of a specific chemical substance as certain reactions are triggered by the binding of reagents to the substance or other related substances.

“Laden” refers to any amount of sample suitable for introduction into a sample receiving port for the purposes of processing a sample using the devices and/or system as described herein.

“Separable” means able to be physically separated or dissociated one thing from the other. For example, the first component and the second component as described herein, are separable.

“Siphon” refers to a pathway, channel, or tube bent to form two legs of unequal length and used to convey liquid or fluid or fluid mixture from a lower level upwards over an intermediate elevation and then down to a lower level.

“Specimen” refers to a sample, portion, or quantity of something such as blood or blood components, body tissue, or any bodily fluid or material that is taken or used for medical or diagnostic testing.

“Thermal contact” refers to contact between two solid bodies wherein energy is exchanged through the process of heat and cool transfer.

“Thermal cycling” as used herein refers to the technology using repeated applications of heating and cooling to cause the DNA strands to separate at higher temperatures and to make new copies of the strands formed from the separated strands at lower temperatures.

“Thin layer” as referred to herein with respect to a fluid or fluid mixture volume refers to the depth or thickness of such a spread volume over a first larger surface area being comparatively small as compared to the greater depth or thickness of such a volume if the same amount were spread over a second smaller surface area.

“Trigger” or “triggered” refers to the initiation or actuation of a physical process or chemical reaction.

“Vertically insert” or “vertical insertion” refers to the vertical, upright, or standing orientation of a cartridge inserted into a second component as described herein such that, within said cartridge, a sample is combined with a lysis buffer at a higher location and elevation than a reaction well, which is located at a lower location and elevation.

“Withdraw” means to remove, take back, or take away something from a particular place or position.

“QR code” is meant to refer to any type of matrix bar code and may be viewable as a mixture of black and white squares and dots.

It is to be understood that any conjugations of the above defined terms shall also be understood to have the same defined meanings as appropriate in the context of the text within which such are written.

### Integrated First and Second Component

After introduction of a sample into the sample receiving port of the first component test cartridge and introduction of the test cartridge into the second component (not necessarily in that order), a push button may be pressed to release buffer (e.g., lysis buffer) from a blister pack). All the sample processing steps may be automated, and thermal cycling may be performed without any further user intervention. Test results may be displayed on a small external touch screen located on an external surface of the second component to provide simplified feedback to the user. Alternatively, test results may be displayed on a computer screen receiving data and information from the system and device described herein.

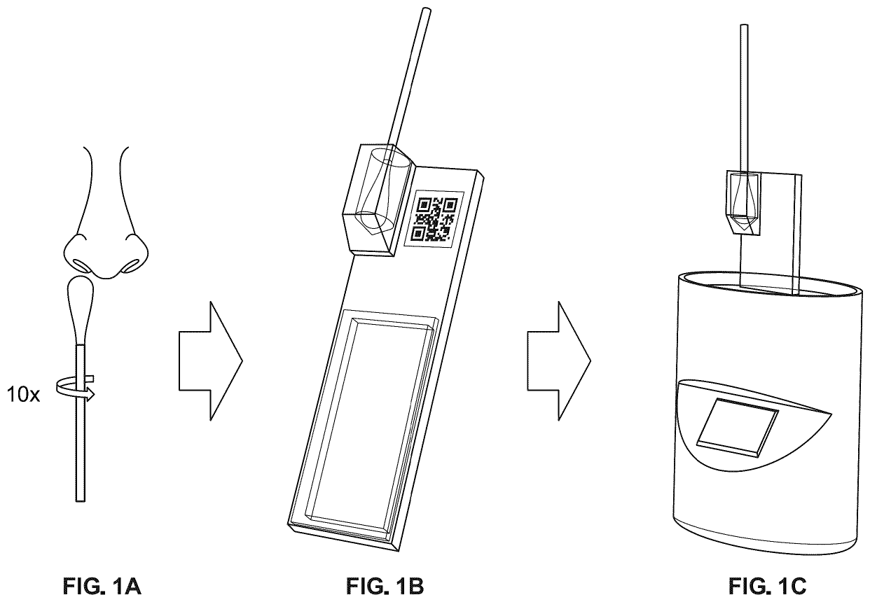

One preferred embodiment of the system and device described herein is shown in FIG. 1. FIG. 1A depicts an embodiment of sample specimen collection. Specifically, FIG. 1A depicts a flocked swab that is to be rotated within the nasal cavity to collect a mucus specimen sample. In alternative embodiments other types of swabs may be used. FIG. 1B depicts a first component test cartridge embodiment that generally shows a sample receiving port, a bar code, and a reaction well located in the lower portion of the vertically oriented test cartridge. FIG. 1C depicts an embodiment of the final form factor of a device disclosed herein wherein the first component test cartridge of FIG. 1B is vertically inserted into the second component comprising an amplification module and an analysis module within a housing. FIG. 1C further depicts an embodiment of the second component wherein the housing includes an external surface display screen.

It is contemplated that, upon loading the sample on the swab and introducing the sample laden swab into the sample receiving port, a user may initiate the test processing and that test feedback may be received via a small screen located on the exterior of the second component. Preferably, the small screen is a touch screen.

Once a test is completed, the test cartridge is disposed of and the two component systems is available for running another sample specimen.

In one preferred embodiment, the final design will be completely enclosed except for the opening used to insert and withdraw first component test cartridges and is simplified as shown, for example, in FIG. 1C.

### First Component

The first component test cartridge includes a sample receiving port and provides for complete processing of a sample within the cartridge.

Complete sample processing includes introduction of a sample material, either directly or via a carrier such as, for example, by a swab, patch, or syringe, etc., to a cartridge sample receiving port. The cartridge sample receiving port forms an integrated part of the first component test cartridge. The cartridge sample receiving port may vary in size depending on the means used to introduce a sample material into the cartridge sample receiving port. The cartridge comprises a buffer region that includes lysis buffer. The lysis buffer can be held in a variety of formats, including but not limited to, for example, a blister pack. It is contemplated that about 400 μl of lysis buffer may be suitable for use with the devices disclosed herein; however, the amount of lysis buffer may vary depending on the final conformation of the components used including, for example, amounts between about 200 μl to 800 μl or greater, about 300 μl to 700 μl, about 400 μl to 600 μl, or about 300 μl to 500 μl. The cartridge may be a microfluidic device.

The lysis buffer is, preferably, pre-loaded into the test cartridge or it may be added by a dropper, or otherwise, into buffer region from the buffer control area of the cartridge. The lysis buffer can be released from the buffer region via a variety of mechanisms triggered from the buffer control area including, for example, by a push button located on an external surface of the cartridge that triggers release of (or rupture, if the lysis buffer is contained in a blister pack) the lysis buffer from the buffer region. Alternatively, the lysis buffer release may be released by the physical introduction of the sample into the sample receiving port. Once the lysis buffer is released from the buffer region, it flows into the sample receiving port and combines with sample introduced into the sample receiving port in a sample and buffer mixing zone. In a preferred embodiment, no external power source is needed to induce the flow of lysis buffer from the buffer region towards the sample receiving port and the sample and buffer mixing zone as this movement of materials is accomplished via gravity.

Transport of the sample and buffer together from the mixing zone and towards the reaction well is accomplished by an inverted siphon mechanism such that the mixture of sample and buffer flow from the mixing zone upward without a pump into a siphon U-turn and then through a downward directed drawing tunnel that causes the sample and buffer mixture to flow downwards under the pull of gravity. The sample and buffer mixture then arrive at and are discharged into the reaction well which is located at a lower level than the sample and buffer mixing zone. Once initiated, the flow of materials via the inverted siphon mechanism continues unaided.

The reaction well may contain one or more reagent materials, including one or more lyophilized reagent materials (e.g., DNA polymerase) that are re-suspended upon introduction of fluid media mixture of sample and lysis buffer. The reaction well has set physical boundaries defining a reaction volume which is generally broad and shallow in nature so as to maximize the thin spread of the reaction mixture in order to facilitate the rapid heating and cooling required for effective thermal cycling. The reaction well can also include one or more reagents formulated to produce a signal that indicates a presence of a target amplicon within the input sample. In a preferred embodiment, the reaction well is pre-loaded with reagent materials. Pre-loading of the reagent materials into the reaction well may occur by spraying or introducing the reagent materials into the reaction well before part ‘A’ and part ‘B’ of the first component test cartridge are combined.

In general, the cartridge has a flattened configuration. As noted above, the flattened cartridge configuration permits uniform rapid cyclical heating and cooling of the reaction well via contact along one or more of the broad flattened external-facing cartridge surfaces with one or more external thermal cycling components. The flattened cartridge configuration also permits use of a minimal reaction volume within the test cartridge and, particularly, within the reaction well.

In one embodiment, the flattened cartridge configuration also comprises one or more narrow side external-facing windows or sufficiently transparent surfaces that may permit transverse excitation of the reaction well and also detection of the PCR.

The cartridge may be single-use and may be disposable. The cartridge may, optionally, include a QR code that may be scanned to identify the test. Alternatively, the cartridge may, optionally, include a near field communication (NFC) tag that may be scanned to identify the test. After a test is completed, the test cartridge is removed from the second component and disposed of or thrown out. Upon completion of a test, the system is available again for running another specimen or sample using another test cartridge and the second component comprising an amplification module and an analysis module.

In a preferred embodiment, RT-PCR assays are run in stand-alone cartridges in a miniaturized format.

Some cartridge designs, as disclosed herein, were developed for the passive control of fluidics using 3D printing technology. In one embodiment, the cartridge is designed for the direct loading of a swab into a sample receiving port. This permits for the loading of specimens collected from a variety of sample matrices including, but not limited to, nasal swabs and fingerpick whole blood.

Cartridge materials and dimensionality allows for optimal fluorescent detection. And the thickness of the cassette plastics, along with reaction well volumes and spread of reaction volume, can be further optimized to enhance both fluorescent detection (excitation and emission) and thermal cycling. In one embodiment, the cartridge dimensions are about 65 mm in length by 30 mm in width, and about 6 mm in thickness along the sides.

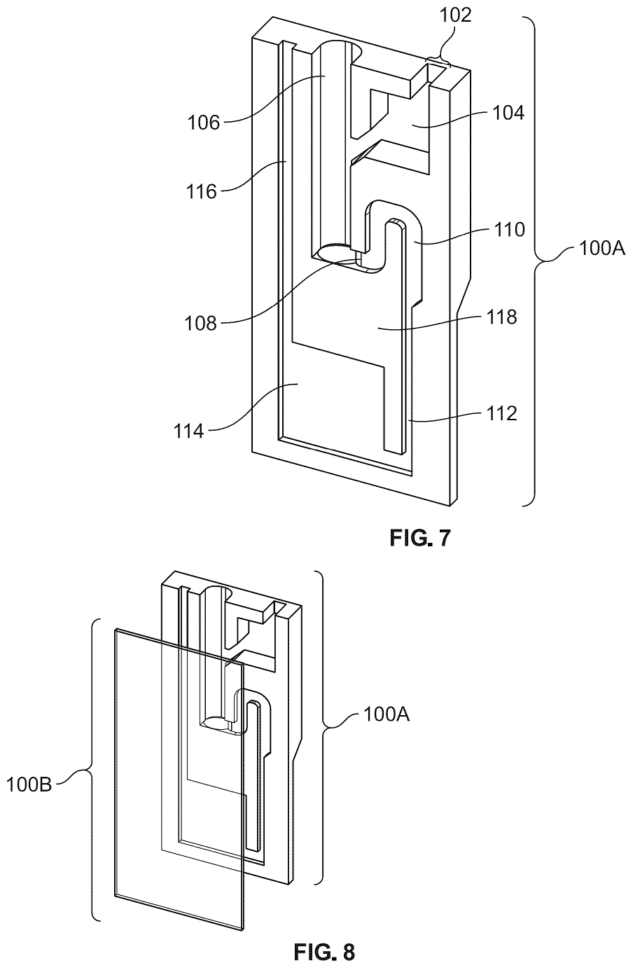

Finalized cartridge designs can be produced using rapid prototyping services with injection molded materials. Injection molding of the cartridges will permit the use of suitable materials in the NAAT device, including but not limited to polycarbonate or polypropylene, for use during the reaction and with the second component. Design iterations of the cartridge may include the addition of, for example, a blister pack and push button or other possible means of providing buffer solution into the sample receiving port. Of course, minimizing required sample volumes and reaction volumes, and the ability to control incubation times, are important cartridge design parameters. It is expected that the reaction well will hold lyophilized reagents (e.g., polymerase, primer, probes, etc.). The buffer and sample mixture will solubilize the reagents in the reaction well. In one embodiment, thermal cycling will be automatically initiated upon any of, for example, the introduction of the first component test cartridge into the second component, upon release of lysis buffer into the sample receiving port, or upon the solubilization of the reagents in the reaction well by the introduction of the sample and lysis buffer mixture. One preferred embodiment of the first component test cartridge used to evaluate fluid flow, thermal cycling and PCR amplification is shown in FIG. 7 (100A) and FIG. 8 (100A and 100B). FIGS. 7 and 100A provides an example interior view of a part ‘A’ of a first component test cartridge 100 that provides controlled fluidic flow within the test cartridge 100. The test cartridge 100 comprises a part ‘A’ and a part ‘B’ which is integrally formed, added, or attached (as shown in FIG. 8), to cover and seal the broad interior cavity surface face 118 of part ‘A’.

As shown in FIG. 7, part ‘A’ of the first component test cartridge 100A includes a buffer control area 102, a buffer region 104, a sample receiving port 106, a buffer mixing zone 108, a siphon U-turn 110, a drawing tunnel 112, a reaction well 114, and a vent 116. Also, included is the interior facing surface 118 which, as noted above, together with part ‘B’ of a first component test cartridge, forms cartridge 100.

Samples may be collected from fingerpick whole blood (˜100 μl) and loaded onto a swab or other piece of material or absorbent pad for handling and then placed into the sample receiving port 106 of the first component test cartridge 100. The swab may be a sterile flocked swab, and may comprise sterile polyester. Alternatively, the swab may comprise foam. The cartridge 100 is inserted into the second component 200 either before or after the sample laden swab is placed into the sample receiving port 106 of the first component 100. A buffer control area 102 may comprise a button (not shown) that is pressed to release buffer from a blister pack or other buffer containing vessel (not shown) but which may be housed in a buffer region 104 onto the swab placed into the sample receiving port 106. The buffer from the blister pack and patient genetic material from the sample laden swab are combined in the buffer mixing zone 108 as the combined sample and buffer material passively migrate via siphon U-turn 110 and drawing tunnel 112 into reaction well 114. The combined sample and buffer material is introduced at a low or the lowest point of the reaction well 114 and access to a vent 116 is provided on an opposite upper or utmost point of the reaction well 114 in order to expel bubbles that might interfere with the thermal cycling and analysis of materials located within the reaction well 114. The reaction well 114 also contains pre-loaded reagent materials such as lyophilized enzymes, primers and probes, that are solubilized and processed together with the sample and buffer mixture to form a reaction volume within the reaction well 114. That is, the reverse-transcription and subsequent PCR amplification steps are performed within the reaction well, and the PCR reaction products formed within the reaction volume within the reaction well 114 are analyzed while the in the reaction well 114.

It is expected that additional modifications to a cartridge such as that shown in FIG. 7 may be required in order to integrate the excitation LED's and photodiode sensors.

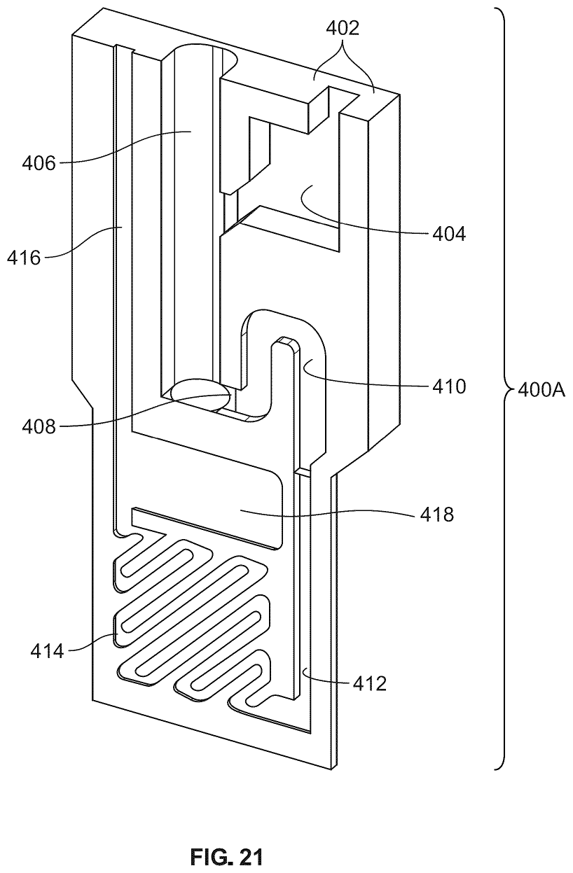

Another preferred embodiment of the first component test cartridge used to evaluate fluid flow, thermal cycling and PCR amplification is shown in FIG. 21 (400A). FIGS. 21 and 400A provides an example interior view of a part ‘A’ of a first component test cartridge 400 that provides controlled fluidic flow within the test cartridge 400. The test cartridge 400 comprises a part ‘A’ and a part ‘B’ (not shown in FIG. 21) which is integrally formed, added, or attached, to cover and seal the broad interior cavity surface face 418 of part ‘A’.

As shown in FIG. 21, part ‘A’ of the first component test cartridge 400A includes a buffer control area 402, a buffer region 404 which may or may not comprise a buffer filled blister pack, a sample receiving port 406, a buffer mixing zone 408, a siphon U-turn 410, a drawing tunnel 412, a reaction well 414, and a vent 416. Also, included is the interior facing surface 418 which, as noted above, together with part ‘B’ of a first component test cartridge, forms cartridge 400. The reaction well 414 as depicted in FIG. 21 differs from the reaction well 114 shown in FIG. 7. The narrow zigzag, or tortuous, course or pathway structure of the reaction well 414 facilitates processing of minimal reaction volumes.

Samples may be collected from fingerpick whole blood (˜100 μl) and loaded onto a swab or other piece of material or absorbent pad for handling and then placed into the sample receiving port 406 of the first component test cartridge 400. The swab may be a sterile flocked swab, and may comprise sterile polyester. Alternatively, the swab may comprise foam. The cartridge 400 is inserted into the second component 400 either before or after the sample laden swab is placed into the sample receiving port 406 of the first component 400. A buffer control area 402 may comprise a button (not shown) that is pressed to release buffer from a blister pack or other buffer containing vessel (not shown) but which may be house in a buffer region 404 onto the swab placed into the sample receiving port 406. The buffer from the blister pack and patient genetic material from the sample laden swab are combined in the buffer mixing zone 408 as the combined sample and buffer material passively migrate via siphon U-turn 410 and drawing tunnel 412 into reaction well 414. The combined sample and buffer material is introduced at a low or the lowest point of the reaction well 414 and access to a vent 416 is provided on an opposite upper or utmost point of the reaction well 414 in order to expel bubbles that might interfere with the thermal cycling and analysis of materials located within the reaction well 414. The reaction well 414 also contains pre-loaded reagent materials such as lyophilized enzymes, primers and probes, that are solubilized and processed together with the sample and buffer mixture to form a reaction volume within the reaction well 414. That is, the reverse-transcription and subsequent PCR amplification steps are performed within the reaction well, and the PCR reaction products formed within the reaction volume within the reaction well 414 are analyzed while the in the reaction well 414.

It is expected that additional modifications to a cartridge such as that shown in FIG. 21 may be required in order to integrate the excitation LED's and photodiode sensors.

### Second Component

The second component comprises an amplification module and an analysis module within a housing that is configured to arrange the amplification module and the analysis module for optimal thermal contact and fluorescent excitation and detection interaction with the reaction volume in the reaction well of the first component test cartridge. The second component may, optionally, initiate, or power on, when a cartridge is introduced or inserted.

The amplification module may comprise one or more thermal cycling elements which are configured to contact at least one broad flattened external-facing cartridge surface and, particularly, the portion of the external-facing cartridge surface that covers the test cartridge reaction well. The one or more thermal cycling elements may be Peltier heating and cooling surfaces and may include one or more fans. In another embodiment, the one or more thermal cycling elements may be altered and, also, additional cooling elements may be added (e.g., Peltier and a fan for use with a heatsink). In still another embodiment, sandwiched Peltier heating and cooling surfaces located on either side of the external-facing cartridge surface that covers the test cartridge reaction well may also be used.

In one embodiment, because the flattened test cartridge configuration provides for contact of the amplification module along one or more of the broad flattened external-facing cartridge surfaces (and, specifically the surfaces corresponding with the reaction well) with one or more external heating and cooling components, the test cartridge provides narrow side external-facing windows or materials with sufficient transparency such that they are adapted to interact with the excitation part and detection part of the analysis module.

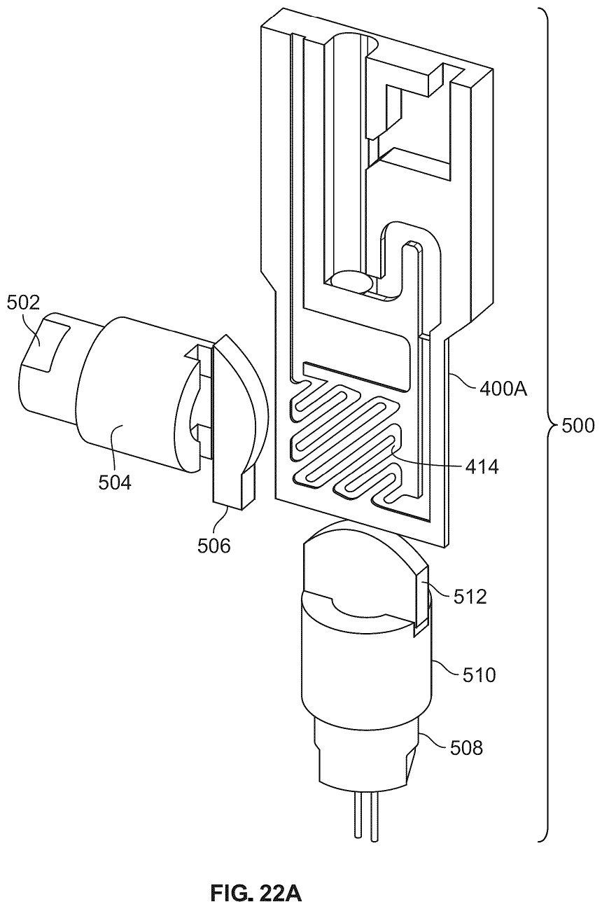

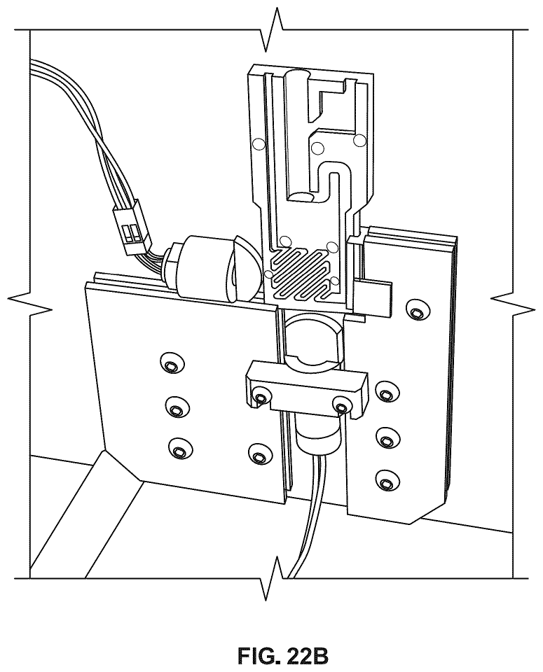

The analysis module comprises an excitation part, such as a light source, to generate a signal (e.g., fluorescence) and a detection part, such as a photodiode or a camera, to receive the signal. The analysis module interacts with the minimal reaction volume in the test cartridge reaction well and may be in communication with a computer. An example of an analysis module is provided in FIG. 22A and FIG. 22B. The excitation part of the analysis module may provide transverse illumination of the minimal reaction volume in the reaction well of the test cartridge by, for example, LED illumination. Thus, the minimal reaction volume in the reaction well of the test cartridge may be illuminated transverse, lateral, or sideways (e.g., 90 degrees) relative to the one or more heating and cooling elements such that an excitation light source may interact with the complete minimal reaction volume. The detection part of the analysis module may provide transverse detection by, for example, a photodiode sensor or a camera. Thus, the detection of signal produced within the minimal reaction volume in the reaction well may be detected transverse, lateral, or sideways (e.g., 90 degrees) relative to the one or more heating and cooling elements.

The analysis module may provide for recordation of the thermal (or isothermal) cycling.

The separately housed amplification module and analysis module may be repeated-use. The separately housed amplification module may, optionally, include a touch screen to display results. The separately housed amplification module may, optionally, be Wi-Fi and/or Bluetooth capable.

### EXAMPLES

Proof-of-principle thermal cycling and successful PCR reactions using both a single Peltier thermal cycling components and a sandwich-style Peltier device using two Peltier thermal cycling components. These reactions have been carried out using off-the-shelf micro reaction chambers. Complete thermal cycling reactions have been performed and recorded.

### 1. Example 1: Thermal Cycle Testing

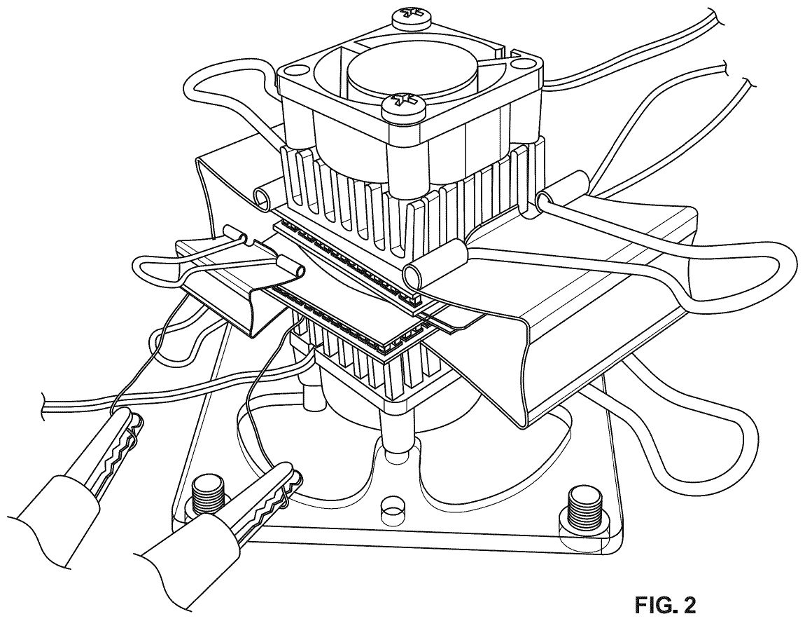

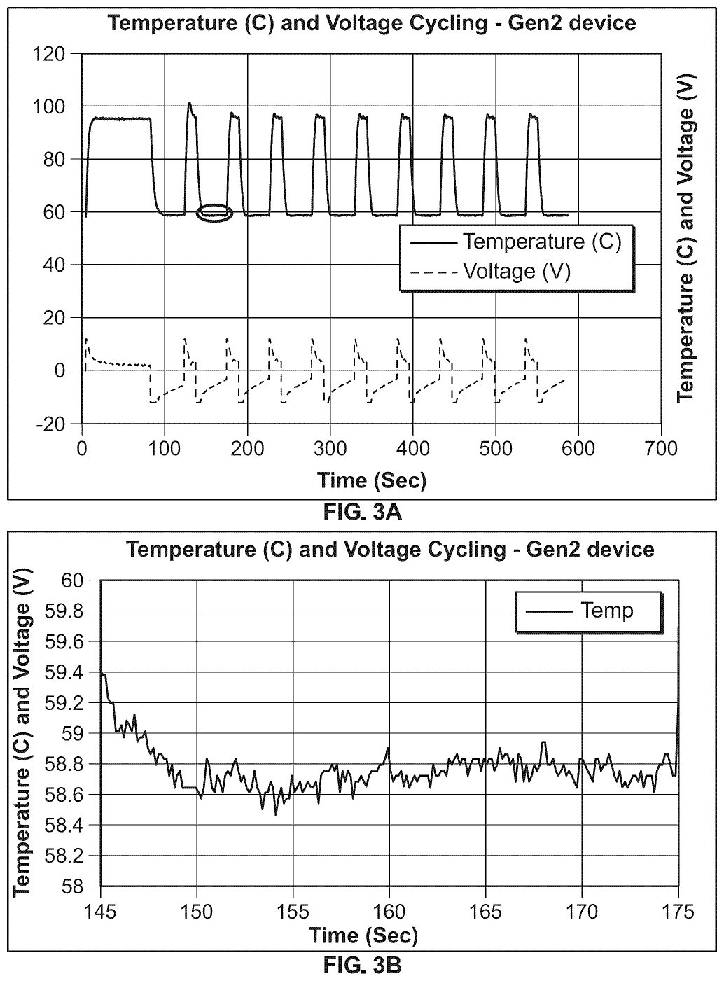

A thermal cycling test was performed using the instrument shown in FIG. 2. As shown in FIG. 3A and FIG. 3B, the precision of the temperature control was held well within ±0.5° C. of the 58.8° C. temperature over a 20 second time period (measured from 150 seconds to 170 seconds), and this is reproducibly generated over a about a 10-cycle PCR run.

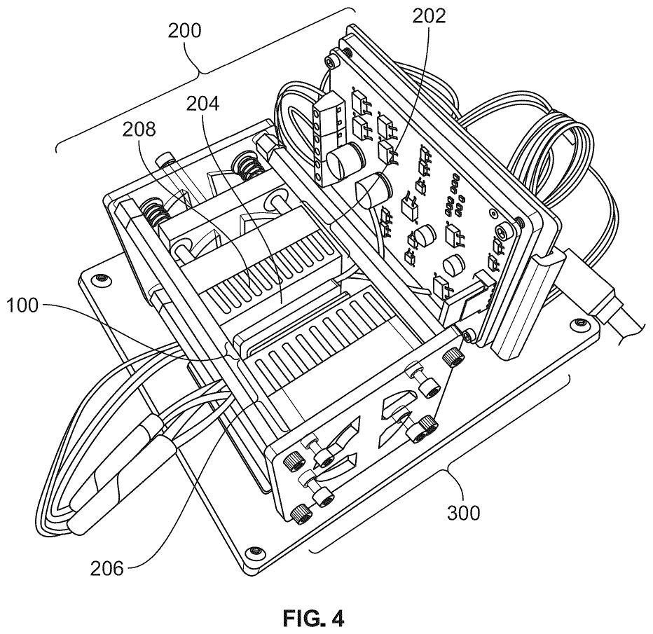

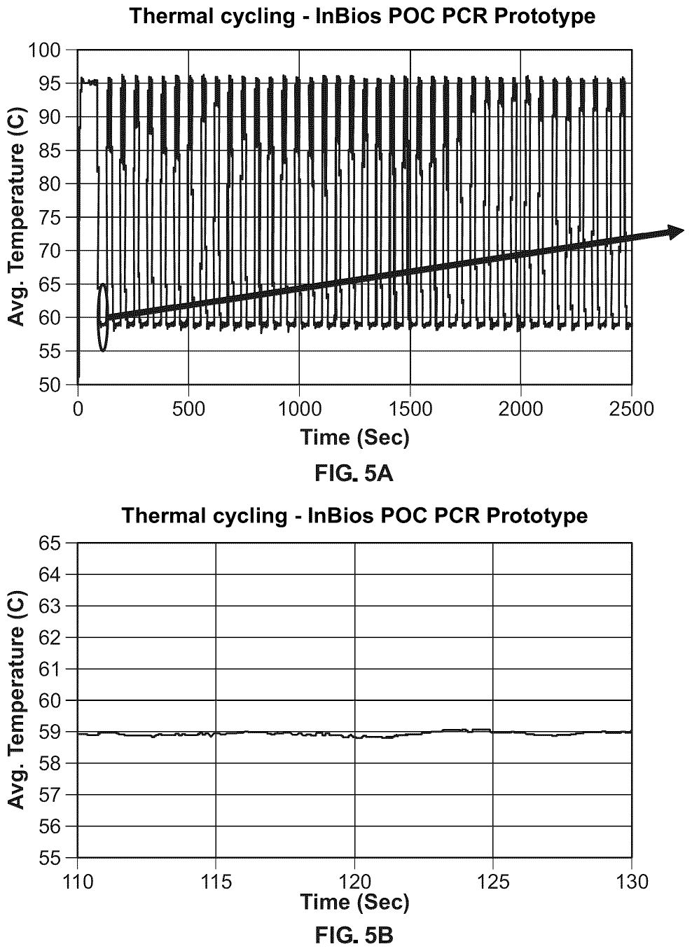

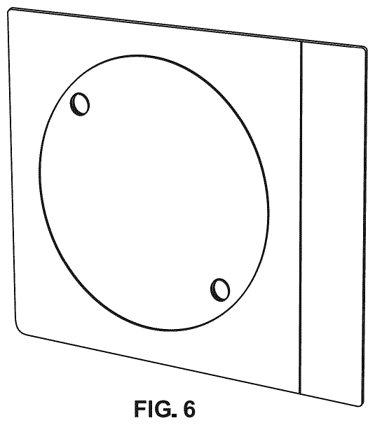

A second thermal cycling test was performed using the instrument shown in FIG. 4. As shown in FIG. 5A and FIG. 5B, the precision of the temperature control was held well within +0.5° C. of the 59° C. set point temperature over a 20 second time period, and this is reproducibly generated over a standard 40-cycle PCR run.

That is, precise temperature monitoring and control of the two-component system described herein provides a hold temperature precision within ±0.5° C. of the set point temperature. In a preferred embodiment, temperature control is achieved using pulse-width modulated (PWM) controlled feedback loops and custom designed software to finely tune the temperature measured at the heating and cooling surface.

### 2. Example 2: Preparation of Primer Sets

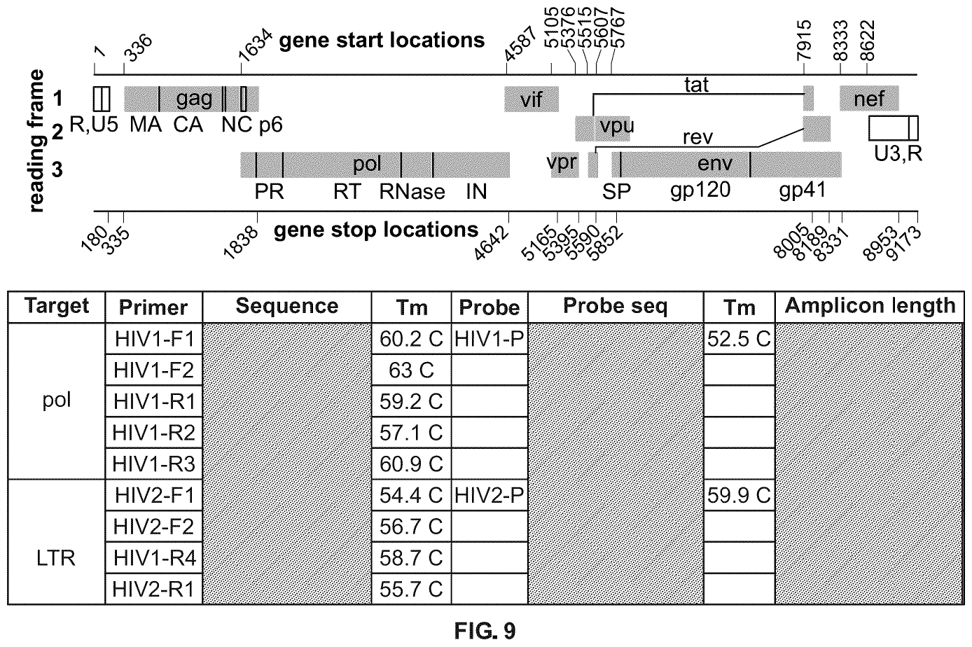

Primers and probes selected for HIV-1 and HIV-2 are indicated in the table of FIG. 9. These designed primers sets detect conserved regions of HIV-1's polymerase gene (pol) and HIV-2's long terminal repeat (LTR). The HIV-1 genome organization is shown with protein coding regions shown as gray boxes; polyprotein domain junctions depicted as solid vertical lines; and gene start and end sites numbered according to NL4-3is. It is noted that this HIV-1 genome organization image is copied from FIG. 1a of Watts J M, et al. Architecture and Secondary Structure of an Entire HIV-1 RNA Genome. Nature. 460, 711-716 (2009).

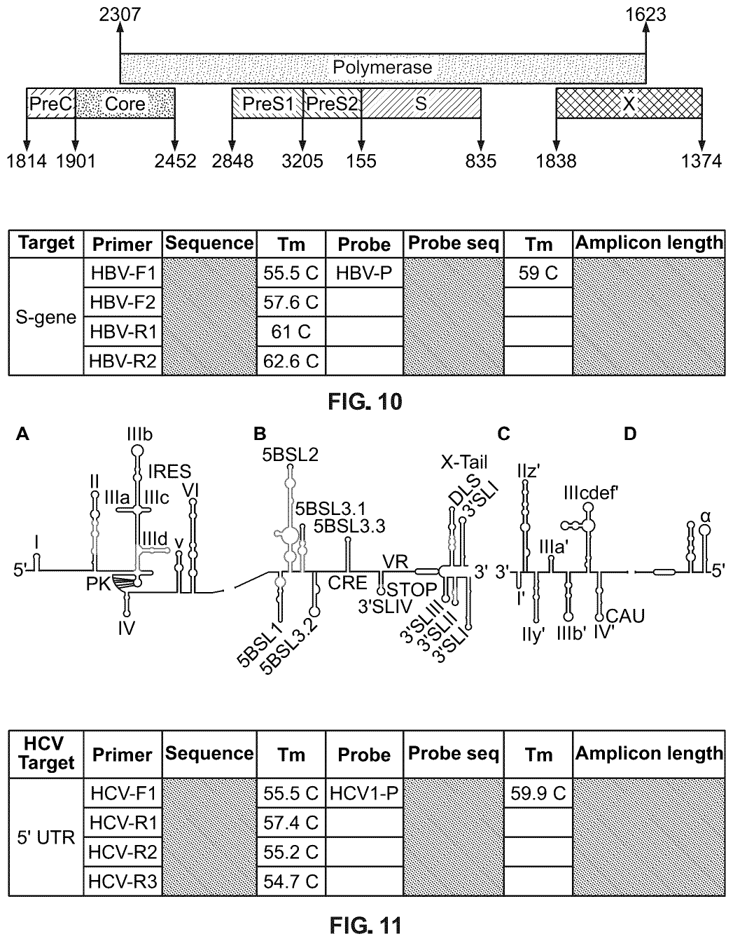

Primers and probes selected for HBV target the S-gene and are indicated in the table of FIG. 10. The image depicting the linear arrangement of overlapping ORFs and viral transcripts is adapted from FIG. 1B of Lamontagne J. Hepatitis B virus and microRNAs: Complex interactions affecting hepatitis B virus replication and hepatitis B virus-associated diseases. World Journal of Gastroenterology. 21, 7375-7399 (2015). As depicted, the transcripts and ORF are aligned with each other according to genomic location and are to scale, and colored boxes on transcripts indicate proposed miRNA target sites.

Primers and probes selected for HCV are indicated in the table of FIG. 11. The primers and probes selected for HCV target the conserved 5′ UTR region of this RNA virus. The image depicting stem-loop structures is copied from FIG. 1 of Fricke M. Conserved RNA secondary structures and long-range interactions in hepatitis C viruses. RNA Journal. 21, 1219-1232 (2015). This image provides an overview of previously known (black) and novel (gray) RNA stem-loop structures of the HCV RNA genome. (A) 5′ UTR and core region SLV and SLVI of the viral plus-strand. (B) CRE region and 3′ UTR of the viral plus-strand. (C) 3′ End of the viral minus-strand. (D) 5′ End of the viral minus-strand.

In silico analysis: Before conducting experiments, bioinformatics tools were employed for primer design and to predict the potential formation of primer dimers. This analysis aimed to identify dimer formation within the same primer pairs designed for targeting the same template, dimer formation between primers and their associated probes, dimer formation between different target sequences, and dimer formation with the probes of other targets. The in-silico analysis indicated that while no oligo hairpin formation was predicted, a few primer dimers were anticipated, although they were not likely to be replicated. Given the substantial number of primers designed for the three targets, it was expected that some primer dimers might form, and experimental verification would be required.

Sequences for HIV-1, HIV-2, Hepatitis B and Hepatitis C were downloaded from the NCBI Virus Database (NCBI Virus (nih.gov)) and aligned using MAFFT v7 (MAFFT—a multiple sequence alignment program (cbrc.jp)). The multiple sequence files were loaded into primer (Bioconductor-rprimer (development version)) and multiple primer and probe sets were designed. This sequences of primers and probes were set aside for further analysis.

The nucleotide sequences for all primers and probes previously designed were analyzed with OligoAnalyzer (Oligo Analyzer (idtdna.com)) to determine the Tm in various buffer and oligo concentrations and to look for hairpin structures. Any nucleotide sequences showing a Tm that was too low or a strong tendency towards 3′ hairpin formation had their primer and probe set removed from the set of potential primers and probes.

The remaining primer and probe sets were combined into putative multiplex sets (one primer and probe set for each target) and the putative multiplex sets were analyzed with both AutoDimer Version 1.0 (AutoDimer download page, nist.gov) and Multiple Primer Analyzer (Multiple Primer Analyzer|Thermo Fisher Scientific-US) for the formation of primer dimers. Multiplex sets that were predicted to produce exponentially amplifying primer dimers due to matches at the 3′ ends of heterodimers were removed from the pool. A final multiplex set was selected based on predicted low primer-dimer formation.

As discussed earlier, there are two types of HIV, HIV-1 and HIV-2, with each bearing many subtypes. More than 97% of all global HIV infections are made up of HIV-1, subtype group M, with HIV-2 responsible for 1-2% of the epidemic. We designed primers sets (see FIG. 9) to detect conserved regions of HIV-1's polymerase gene (pol) and HIV-2's long terminal repeat (LTR).

### 3. Example 3: Use of System to Target Influenza B Compared to Commercially Available System

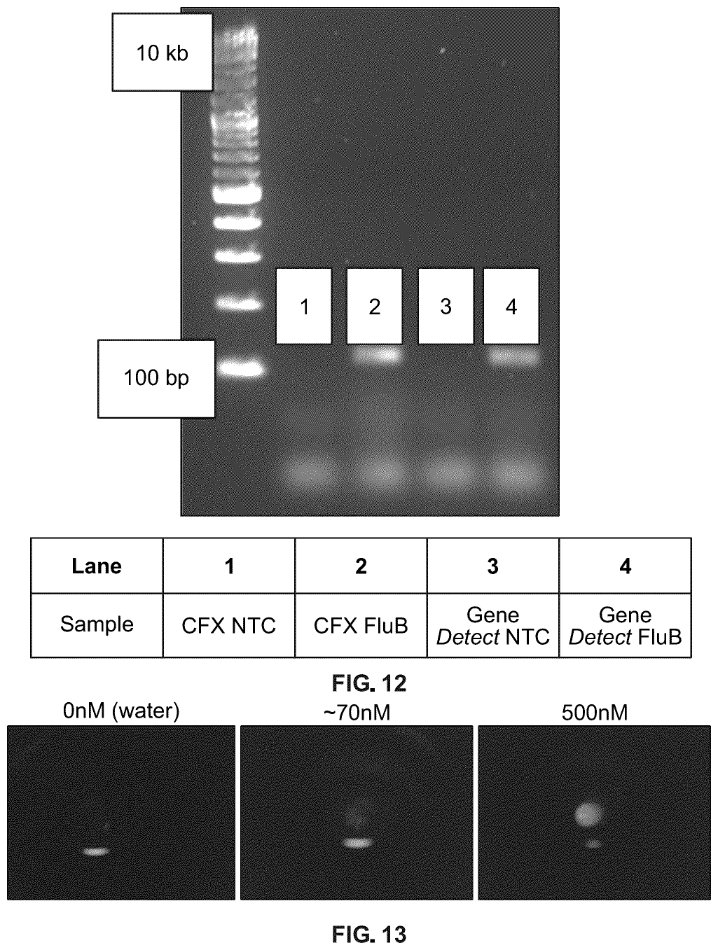

Testing was performed with the device using a simple PCR assay created to target the influenza B (flu B) matrix gene. The flu B amplicon PCR proof-of-principle testing detects an amplicon of length 139 bp and is amplified using two forward primers, one reverse primer and a FAM labelled probe. The oligos are as shown here in Table 1 and the results are shown in FIG. 12.

TABLE 1
The primer used for targeting the influenza B matrix gene.
Primer/Probe

FluB_Matrix-F
FluB_Matrix-F2
FluB_Matrix-R
FluB_Matrix-P

To perform the PCR reaction, 4 uM of each oligo were assembled in a tube prior to preparing the reaction mix. The reaction components for the PCR contains 9 μl of water, 5 μl of Luna® Probe One-Step RT-qPCR 4× master mix, 1 μl of primer/probe mix and 5 μl of amplicon in each 20 μl reaction. The amplicon is made from the 1:500-fold dilution of PCR products from a previous PCR reaction. Subsequently, PCR tubes (for the Bio-Rad CFX) or test cartridges (for the device described herein) were loaded into the thermocycler for the amplification step of the PCR. The initial denaturation step is set at 95° C. for one minute, followed by the standard denaturation step, which is set at 95° C. for 10 seconds. Extension and annealing steps are combined and occur at 59° C. for 30 seconds. The number of cycles is set for 40 for denaturation, extension and annealing steps.

The same reactions for diluted amplicon product of influenza B target and no template control (NTC) materials were run in both a Bio-Rad CFX96 machine (CFX) and the device described herein (referred to as Gene Detect). The PCR products were analyzed via SDS-PAGE gel. As can be seen in FIG. 12, the PCR product for influenza B is present in both the CFX device and the Gene Detect device described herein at the expected size (139 bp) while the NTC showed no appreciable amplification.

### 4. Example 4: Performance Matching Study

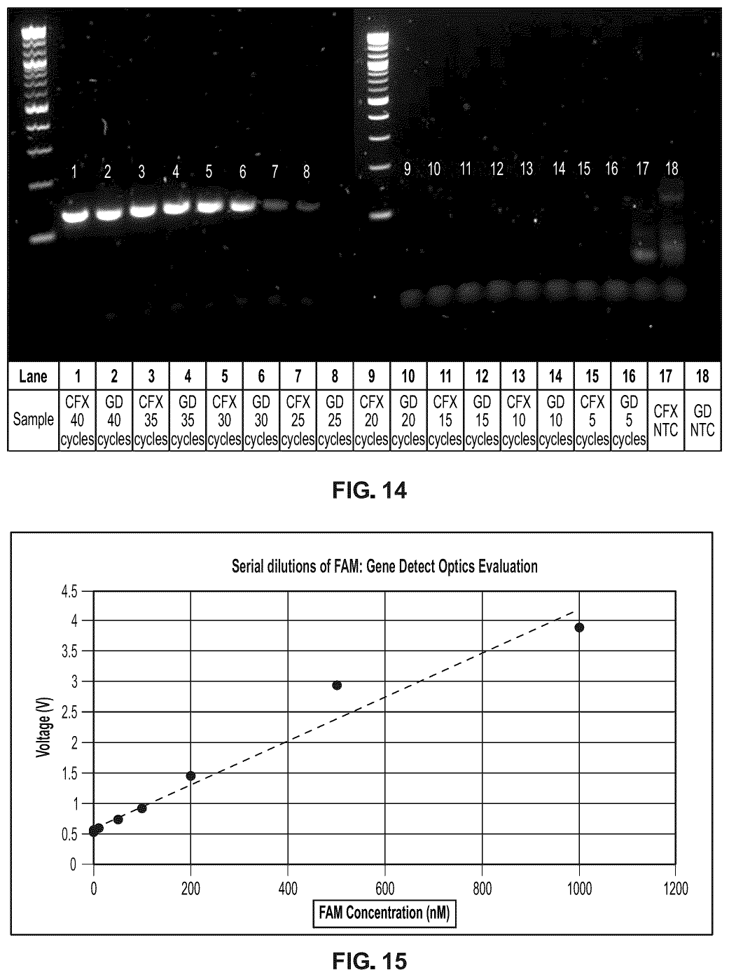

To further ensure that the PCR product generated with the device described herein matched the performance of the commercially available Bio-Rad CFX device, the influenza B PCR assay was performed with varying cycle counts, ranging from 5, 10, 15, 20, 25, 30, 35 and 40 cycles in each device. The PCR products that were generated using the CFX device and the device described herein were analyzed side-by-side via a 3% agarose gel to qualitatively compare the results as summarized in FIG. 14. It is noted that no template control (NTC) was performed for 40 cycles and are shown in lanes 17 and 18.

### 5. Example 5: Real-Time Fluorescent Measurements

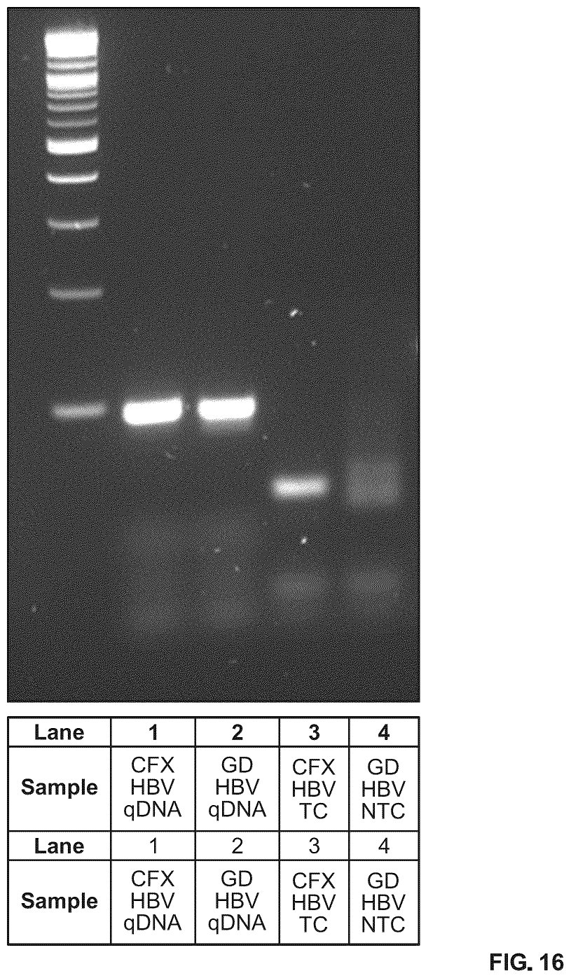

An additional critical step for on-device, real-time measurement of the PCR product from the device described herein is the integration of on-board, real-time fluorescent measurements. This requires 1) the appropriate excitation light source and filter sets and 2) the corresponding emission filter sets and sensors. The complete integration of the excitation and emission filters is dependent upon the final configuration of the cartridge design; it is expected that once this design if finalized, the illumination and real-time fluorescent measurements will be included as part of the device described herein. To ensure that the initial selected filter sets and illumination sources were appropriate for the FAM fluorophore, we ensured that serial two-fold dilutions of the fluorophore concentration resulted in a linear relationship of the measured voltage at the photodiode, as seen in FIG. 15.

### 6. Example 6: Singleplex Detection

The primers and probes for HBV, HCV and HIV were first tested under optimum conditions using both a standard laboratory real-time PCR machine (C1000 Touch Thermal Cycler with a CFX96 Optical Reaction Module, hereafter abbreviated as CFX) and the device described herein. This consisted of testing the primers and probes in single plex using New England BioLab's Luna® Probe One-Step RT-qPCR 4× Mix with UDG as a RT-qPCR master mix with known positive and negative controls. The positive controls used were either quantitative synthetic material from ATCC (HBV: VR-3232SD, HCV: VR-3233SD) or extracted viral culture from Zeptometrix (HIV-1: HIV Type 1 (Strain: IIIB) Culture Fluid). The negative control was PCR grade water. The reactions were run in a 96-well optical PCR plate for the CFX96 and were injected into a reaction module for the device described herein. After the PCR reactions were complete, samples were run on a 3% agarose gel with 1 μL of SybrSafe added to each 10 μL of the gel for visualization. Due to limitations on the size of the gel, HBV samples were run on a separate gel than HIV and HCV.

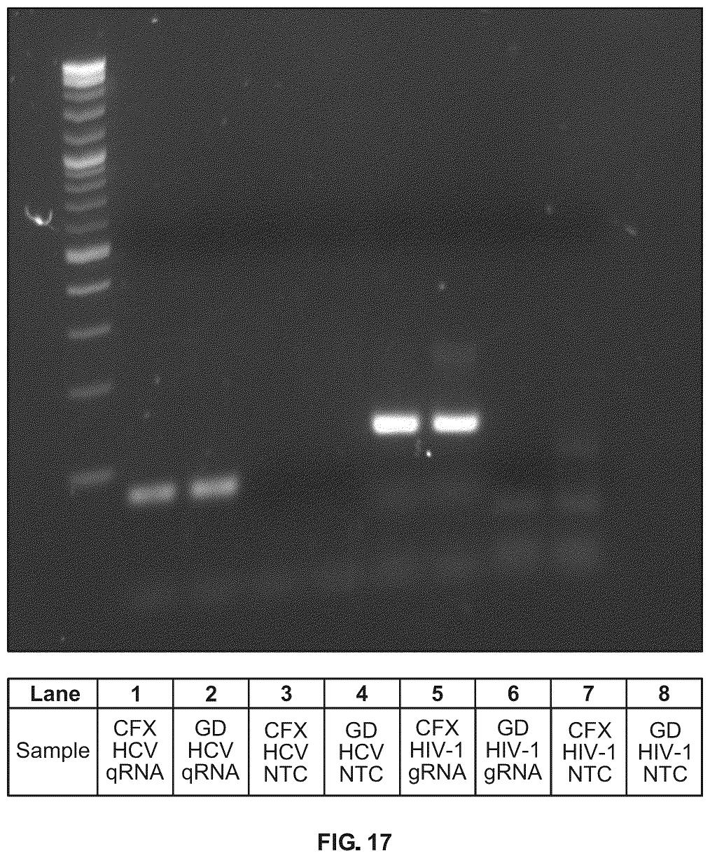

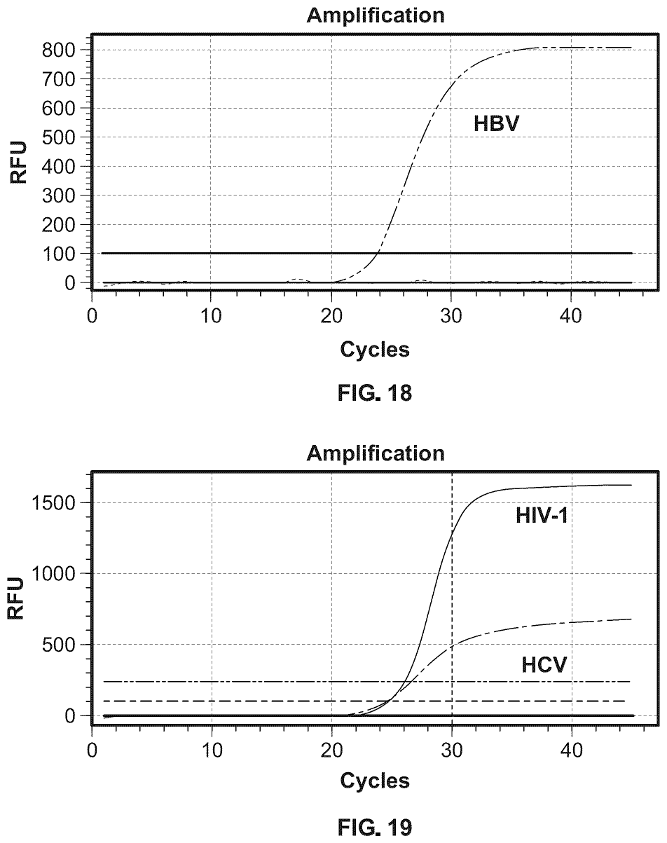

HBV single plex reactions are shown in FIG. 16 while the HCV and HIV-1 single plex reactions are shown in FIG. 17. Corresponding CFX trace is shown in each of FIG. 18 (HBV) and FIG. 19 (HCV and HIV-1), respectively.

### 7. Multi-Plex Detection: HBV, HCV, HIV-1

Efforts were made for the successful multiplexing reverse transcription PCR (RT-PCR) detection of HBV, HCV, and HIV. These efforts included initial testing for multiplex detection and screening for polymerase activity in the presence of whole blood. RT-PCR testing was performed using both the Bio-Rad CFX96 instrument and the device described herein.

For hepatitis B virus (HBV), Hepatitis C virus (HCV) and human immunodeficiency virus (HIV-1) multiplexed detection, fingerpick whole blood can be collected and either transferred directly or directly absorbed into the sterile polyester swab prior to placing into the test cartridge. The cartridge will hold a blister pack that will be released with a single push button.

Progress of the NAAT can be monitored via multicolor fluorescent detection and quantified as a function of time with each cycle. In this manner, it is expected that an estimated Ct (cycle) will be determined for each of HBV, HCV, and HIV with the assay. It is noted that the values may only be considered semi-quantitative due to potential variability in specimen volume added and operator training or use.

The primers and probes selected for HBV target the S-gene and are indicated in FIG. 10. The primers and probes selected for HCV target the conserved 5′ UTR region of this RNA virus and are indicated in FIG. 11.

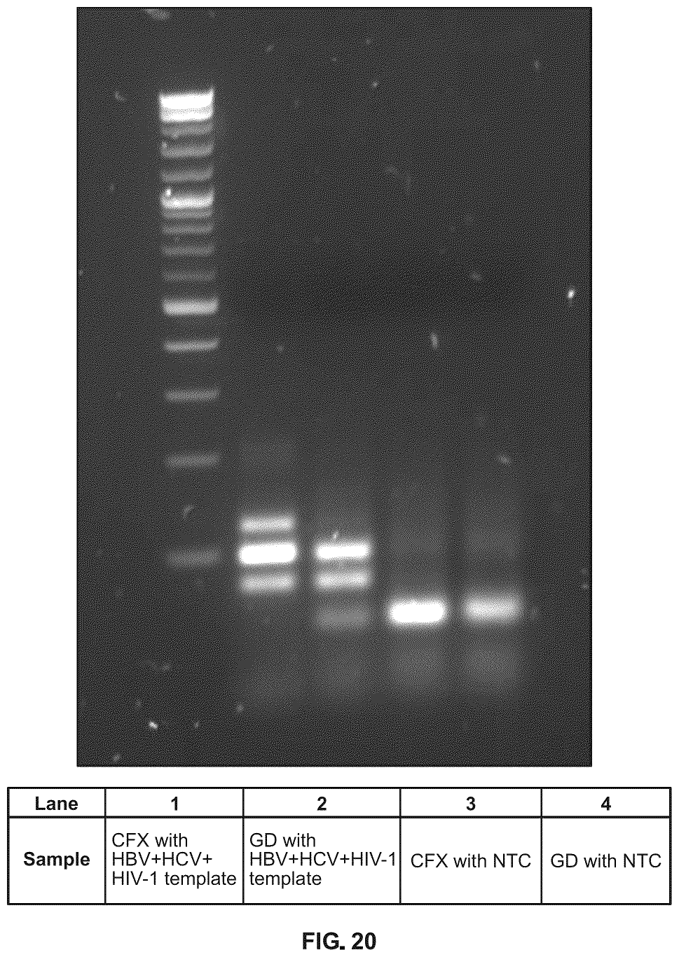

Multiplex testing for HBV, HCV and HIV was performed on both the CFX and the device described herein. The testing was performed as described in the single plex testing except that all primer and probes to be used were mixed in the same multiplex reaction. As can be seen in FIG. 20, the multiplex reaction worked for the detection of HBV, HCV and HIV with the CFX. However, when run on the device described herein, the device only showed the HBV and HCV bands corresponding to the targets when multiple target templates were added to the same reaction. It is expected that primer optimization, temperature optimization, etc., may be used to improve multiplex detection of the device described herein with respect to multi-template samples.

### 8. FAM Concentration Vs. Photodiode Output

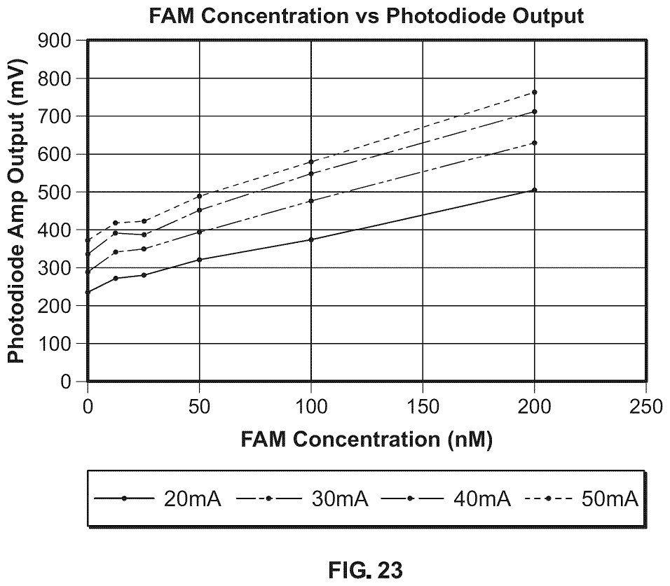

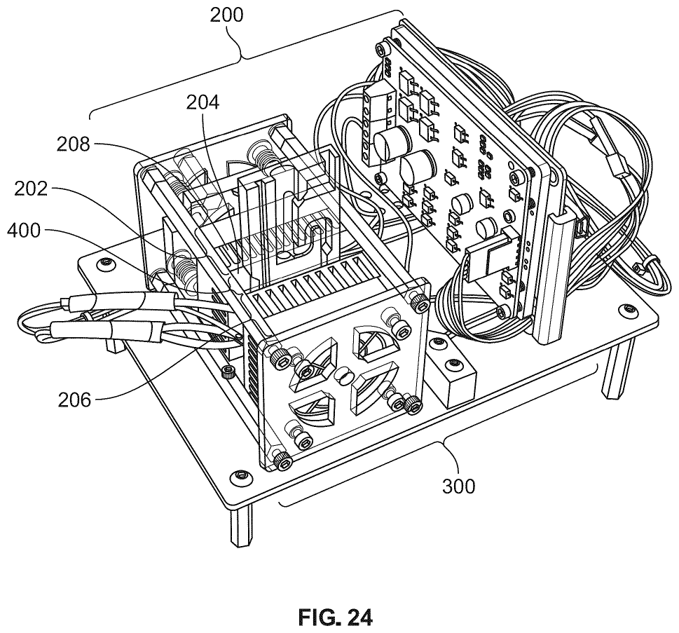

The system depicted in FIG. 22B was used to generate the data shown in FIG. 23. The four lines in FIG. 23 correspond to four different LED currents for different intensity of light. For the data points, the X axis represents the concentration (in nanomolar) of FAM fluorescent solution loaded in the cartridge. The Y axis represents the output from the photodiode (in millivolts). This system lets us know the level of fluorescence of the solution in the cartridge at any point during the PCR process, by monitoring the output voltage.

The present application claims priority to U.S. Provisional Patent Application No. 63/597,611 filed on Nov. 9, 2023, the entire contents of which are incorporated herein by reference.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the breadth and scope of a disclosed implementation should not be limited by any of the above-described implementations, but should be defined only in accordance with the following claims and their equivalents.

1. A test cartridge comprising a controlled fluid pathway connecting a sample receiving port to a siphon U-turn.

2. The test cartridge of claim 1, further comprising a buffer mixing zone between the sample receiving port and the siphon U-turn.

3. The test cartridge of claim 1, wherein the controlled fluid pathway also connects the siphon U-turn to a drawing tunnel, and the drawing tunnel connects to a reaction well.

4. The test cartridge of claim 1, further comprising at least one of: a buffer region; a buffer control area; pre-loaded lyophilized reagent materials; and a vent connected to the reaction well.

5. The test cartridge of claim 1, wherein the test cartridge comprises a reaction well sized and shaped to facilitate a thin spread of a solubilized reaction mixture over an internal surface area and, optionally, wherein the reaction well comprises a narrow zigzag pathway structure.

6. The test cartridge of claim 1, wherein a sample introduced to the sample receiving port is automatically fully processed.

7. A two-component device comprising:
a. a first component test cartridge; and
b. a second component comprising an amplification module and an analysis module.

8. The device of claim 7, wherein the first component and the second component are readily combined.

9. The device of claim 7, wherein the first component and the second component are readily separable.

10. The device of claim 7, wherein the first component is a single-use test cartridge.

11. The device of claim 7, wherein the second component further comprises a separate housing.

12. The device of claim 7, wherein the first component test cartridge comprises a controlled fluid pathway comprising a siphon U-turn.

13. The device of claim 7, wherein the first component test cartridge comprises a sample receiving port.

14. The device of claim 7, wherein the first component test cartridge can be inserted into the second component in a generally vertical orientation.

15. The device of claim 7, wherein the second component amplification module includes one or more thermal cycling components.

16. The device of claim 7, wherein the first component test cartridge comprises a reaction well and, optionally, wherein the reaction well comprises a narrow zigzag pathway structure.

17. The device of claim 7, wherein the reaction well of the first component test cartridge is configured to coincide with one or more thermal cycling components included in the second component amplification module.

18. The device of claim 7, wherein temperature control is maintained within ±0.5° C. of a set point temperature over a 20 second time period, and this is reproducibly generated over a standard 40-cycle PCR run.

19. A method of using the test cartridge of claim 1 to generate PCR test results in either a single plex or multiplex format.

20. A method of using the device of claim 8 to generate PCR results in either a single plex or multiplex format.
